(19) United States
(12) Patent Application Publication
(10) Pub. No.: US 2014/0206085 A1
(43) Pub. Date: Jul. 24, 2014

(54) CASSETTE INCLUDING PROMOTER SEQUENCE OF TARGET GENE AND METHOD OF GENE MANIPULATION USING THE SAME

(71) Applicants: Chung-Ang University Industry Cooperation Foundation, Seoul (KR); Samsung Electronics Co., Ltd., Suwon-si (KR)

(72) Inventors: JAE-YOUNG KIM, SUWON-SI (KR); JIN-KYU KANG, DAEGEON (KR); CHANG-DUK KANG, GWACHEON-SI (KR); SUNG-SOO KIM, HWASEONG-SI (KR); JU-YOUNG LEE, DAEGU (KR); HYUN-AH KANG, SEOUL (KR); JAE-CHAN PARK, YONGIN-SI (KR); HUI-SUB LIM, SEOUL (KR); JIN-HO CHOO, SEOUL (KR)

(73) Assignees: Chung-Ang University Industry Cooperation Foundation, Seoul (KR); Samsung Electronics Co., Ltd., Suwon-si (KR)

(21) Appl. No.: 14/159,718

(22) Filed: Jan. 21, 2014

(30) Foreign Application Priority Data
Jan. 22, 2013 (KR) 10-2013-0007091

(51) Int. Cl. C12N 15/81

(52) U.S. Cl. CPC C12N 15/81
USPC 435/471; 435/320.1

(57) ABSTRACT

Provided is a cassette for deleting a target gene comprising (a) a promoter-specific homologous region having a sequence identity to a portion of a promoter region of the target gene, wherein the degree of sequence identity is sufficient to drive homologous recombination therebetween, (b) a marker gene operably linked to the promoter-specific homologous region, and (c) a gene-specific homologous region adjacent to 3′-end of the marker gene and having a sequence identity to at least a portion of the target gene, wherein the degree of sequence identity is sufficient to drive homologous recombination therebetween.

### CROSS-REFERENCE TO RELATED APPLICATIONS

This application claims the benefit of Korean Patent Application No. 10-2013-0007091, filed on Jan. 22, 2013 in the Korean Intellectual Property Office, the entire disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 18,392 Byte ASCII (Text) file named “713499_ST25.TXT,” created on Jan. 20, 2014.

### BACKGROUND

1. Field

The present disclosure relates to cassettes including promoter sequences of target genes and methods of gene manipulation using the cassettes.

2. Description of the Related Art

Metabolic engineering refers to a series of experiments and prediction technologies for changing metabolic properties of cells or bacterial strains into desired properties by adding a new metabolic pathway or by removing, amplifying, or changing an existing metabolic pathway by using gene manipulation technology. Modifying an existing biological system into a more efficient system suitable for a purpose, or developing a new biological system by combining the components of living things in various ways based on the technologies, may be anticipated.

Through genetic manipulation technology, a specific gene may be removed or added such that a target cell may have desired characteristics. A technology for efficiently selecting genetically modified target cells using markers is needed for a successful manipulation of the genes.

When a specific gene is to be deleted, homologous recombination is generally used, wherein a DNA fragment to be substituted with a target gene is integrated to a chromosome. Conventionally, markers were expressed even when the DNA fragments were randomly integrated to the chromosome. In particular, a technology for distinguishing cells where a target gene is precisely targeted is needed for cells having a low genetic manipulation efficiency.

### SUMMARY

Provided are cassettes for deleting target genes. The cassettes comprise (a) a nucleotide region having a sequence identity to a portion of a promoter of the target gene (i.e., a promoter-specific homologous region), (b) a marker gene operably linked to the promoter-specific homologous region, and (c) a nucleotide region having a sequence identity to at least a portion of the target gene (i.e., a gene-specific homologous region), which is located adjacent to 3′-end of the marker gene.

Provided are methods of preparing cells where the target gene has been deleted by using the cassette.

Additionally provided are methods of isolating cells where the target gene has been deleted by using the cassette. In one particular embodiment, the method comprises introducing the cassette comprising (a) a promoter-specific homologous region, (b) a fluorescent protein gene operably linked to the promoter-specific homologous region, and (c) a gene-specific homologous region, which is located adjacent to 3′-end of the fluorescent protein gene, into a host cell; and isolating cells expressing fluorescence among cells where the cassette has been introduced.

### BRIEF DESCRIPTION OF THE DRAWINGS

These and/or other aspects will become apparent and more readily appreciated from the following description of the embodiments, taken in conjunction with the accompanying drawings.

FIGS. 1A-B schematically illustrate a deletion of a gene through homologous recombination with a cassette. FIG. 1A illustrates a random insertion of a cassette into a genome, and FIG. 1B illustrates targeting the cassette to a target gene in the genome, wherein P denotes a promoter region. While a reporter gene is not expressed when the cassette is randomly inserted, the reporter gene may be expressed under the integrated promoter produced by homologous recombination when the cassette is targeted to the target gene.

FIG. 2 schematically illustrates a deletion cassette, which is for the deletion of a ScADE2 (S. cerevisiae phosphoribosylaminoimidazole carboxylase) gene, wherein P denotes a promoter region and T denotes a transcription terminator.

FIGS. 3A-C are histograms illustrating the results of a fluorescence-activated cell sorting (FACS) measurement of a cell where the deletion cassette has been introduced. FIG. 3A shows a FACS measurement of a cell without a cassette, FIG. 3B shows a FACS measurement of a cell where a cassette has been targeted to the ScADE2, and FIG. 3C shows a FACS measurement of a cell where a cassette has been randomly inserted.

FIGS. 4A-C are images illustrating a cell where the deletion cassette has been introduced, observed by using a fluorescent microscope. FIG. 4A illustrates a cell without introducing a cassette, FIG. 4B illustrates a cell where a cassette has been targeted to a ScADE2 gene, and FIG. 4C illustrates a cell where a cassette has been randomly inserted.

FIG. 5 schematically illustrates a PCR analysis for identifying a deletion of a ScADE2 gene using the cassette.

### DETAILED DESCRIPTION

Additional aspects will be set forth in part in the description which follows and, in part, will be apparent from the description, or may be learned by practice of the presented embodiments.

The present invention employs homologous recombination, which is a type of genetic recombination in which nucleotide sequences are exchanged between two similar or identical nucleotide sequences.

According to an aspect of the present invention, there is provided a cassette for deleting a target gene. The cassette comprises, consists essentially of, or consists of (a) a nucleotide region that is homologous (similar or identical, such as in function or percent identity) to a portion of a promoter of a target gene (herein referred to as a “promoter-specific homologous region”), wherein the degree of sequence identity is sufficient to drive homologous recombination therebetween, (b) a marker gene operably linked to the promoter-specific homologous region, and (c) a nucleotide region that is homologous (similar or identical) to a region that is at least a portion of the target gene (herein referred to a gene-specific homologous region”), which is located adjacent to 3′-end of the marker gene, wherein the degree of sequence identity is sufficient to drive homologous recombination therebetween.

The deletion cassette refers to a DNA module having a structure for directly deleting a target gene by using homologous sequences. The term “homologous” as used herein refers to a degree of sequence identity or similarity with respect to a target sequence. For example, the homologous regions can contain a degree of sequence identity or similarity greater than or equal to 90% or 95% (e.g., 91%, 92%, 93%, 94%, 95%, 96%, 97%, 99%, or 100%) to the corresponding sequence.

In one embodiment, the promoter-specific homologous region may include sequences with a sequence identity or similarity of, for example, 90% or more (e.g., 95% or more, 96% or more, 97% or more, 98% or more, 99% or more, or 100%) to a portion of a promoter sequence of the target gene. The portion of a promoter region of the target gene may comprise a region of 40 to 200 nucleotides, 40 to 150 nucleotides, 40 to 100 nucleotides, or 40 to 80 nucleotides in a direction from a 3′-terminus to a 5′-terminus of a promoter region of the target gene.

“Operably linked to a promoter-specific homologous region” refers to a linkage between the promoter-specific homologous region and a marker gene in such a manner that the marker gene may be expressed by an integrated promoter when the promoter-specific homologous region is integrated into the target gene promoter. For example, when a recombination occurs between the promoter-specific homologous region and a non-homologous sequence, the marker gene operably linked to the promoter-specific homologous region may not be expressed. In contrast, when a recombination occurs between the promoter-specific homologous region and a portion of a promoter of the target gene, the marker gene operably linked to the promoter-specific homologous region may be expressed.

The marker gene may be, for example, an antibiotic resistant gene or a fluorescent protein gene. The antibiotic resistant gene may be selected from the group consisting of, for example, a kanamycin gene, a chloramphenicol gene, and a tetracycline gene. Additionally, the fluorescent gene may be selected from the group consisting of, for example, a yeast-enhanced green fluorescent protein (yEGFP) gene, a green fluorescent protein (GFP) gene, a blue fluorescent protein (BFP) gene, and a red fluorescent protein (RFP) gene.

The marker gene may include, for example, a transcription terminator. The transcription terminator may be selected from the group consisting of, for example, a transcription terminator of a CYC1 (iso-1-cytochrome C) gene, a transcription terminator of a TRP1 (phosphoribosyl-anthranilate isomerase) gene, and a transcription terminator of an ADH1 (alcohol dehydrogenase 1) gene.

The gene-specific homologous region may include sequences with an identity of, for example, 90% or more (e.g., 95% or more, 96% or more, 97% or more, 98% or more, 99% or more, or 100%) to at least a portion of the target gene. The portion of the target gene may comprise a region of, for example, 40 nucleotides to 500 nucleotides, 40 nucleotides to 150 nucleotides, 40 nucleotides to 100 nucleotides, or 40 nucleotides to 80 nucleotides of the target gene.

In the cassette, the gene-specific homologous region may be located adjacent to 3′-end of the marker gene. The gene-specific homologous region can comprise a sequence that is homologous to at least a portion of the target gene and/or a sequence that is homologous to the sequence adjacent to 3′-end of the target gene.

According to another aspect of the present invention, there is provided a method of preparing a cell where a target gene has been deleted. The method comprises, consists essentially of, or consists of introducing a cassette for deleting a target gene into a host cell; and identifying the cell where the target gene has been deleted among cells where the cassette has been introduced (e.g., by assaying for the expression of the marker gene).

The method may further comprise preparing a cassette for deleting a target gene, the cassette comprising (a) a promoter-specific homologous region, (b) a marker gene operably linked to the promoter-specific homologous region, and (c) a gene-specific homologous region, which is located adjacent to 3′-end of the marker gene. The preparation of the cassette for deleting a target gene may comprise, for example, obtaining an amplified product by amplification using a polynucleotide including the marker gene as a template, a forward primer comprising a 5′-terminal region sequence of the marker gene and a sequence of the gene-specific homologous region, and a reverse primer comprising a 3′-terminal region sequence of the marker gene and a sequence of the gene-specific homologous region. The template polynucleotide may be, for example, a plasmid comprising the marker gene.

The forward primer may include, for example, a sequence that is identical or complementary to 10 nucleotides to 30 nucleotides in a direction from a 5′-terminus to a 3′-terminus of the marker gene, at a 3′-terminal site of the primer. Also, the forward primer may include, for example, a promoter-specific homologous region of the cassette at a 5′-terminal site of the primer.

The reverse primer may include, for example, a complementary sequence to 10 nucleotides to 30 nucleotides in a direction from a 3′-terminus to a 5′-terminus of the marker gene, at a 3′-terminal site of the primer. Also, the reverse primer may include, for example, a gene-specific homologous region sequence of the cassette at a 5′-terminal site of the primer.

The host cell may be, for example, yeast. The yeast may be selected from the group consisting of, for example, Saccharomyces cerevisiae, Hansenula polymorpha, Pichia pastoris, Kluyvermyces fragilis, Kluveromyces lactis, Kluyveromyces marxianus, and Schizosaccharomyces pombe. Introducing the cassette into the host may be performed by using any suitable method, such as microinjection, calcium phosphate sedimentation, electroporation, liposome-mediated transfection, DEAE-dextran transfection, and gene bombardment.

The cassette introduced into the host cell may be, for example, integrated into a chromosome of the host cell through homologous recombination. Hence, a target gene may be deleted due to homologous recombinations between the promoter-specific homologous region of the cassette and its target site, and between a gene-specific homologous region of the cassette and its target site.

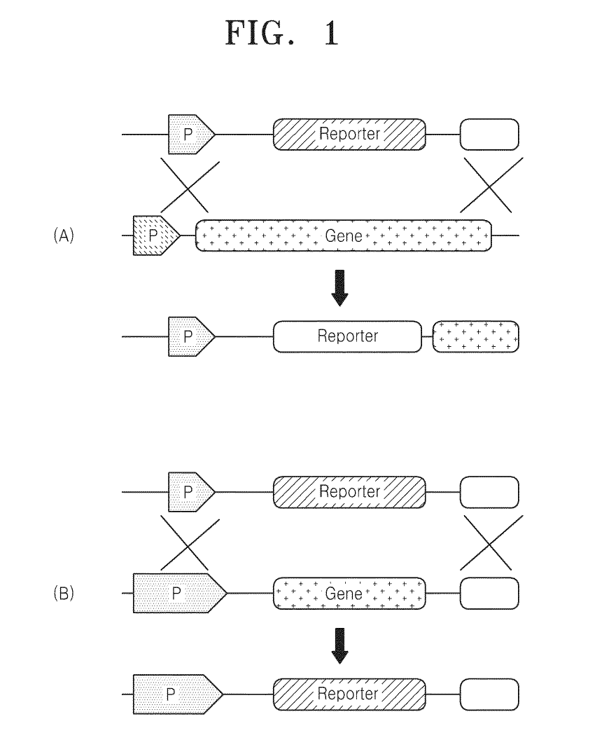

FIG. 1 schematically illustrates a deletion of a gene through a homologous recombination of a cassette. FIG. 1A illustrates a random insertion of a cassette to a genome, and FIG. 1B illustrates targeting the cassette to a target gene in the genome. While a reporter gene is not expressed when the cassette is randomly inserted, the marker (reporter) gene may be expressed under the integrated promoter produced by homologous recombination when the cassette is targeted to the target gene.

A deletion of the target gene may be, for example, identified by a protein expressed from a marker gene that is integrated into a chromosome of the host cell under the integrated promoter. The marker gene may be, for example, an antibiotic resistant gene as described above. When the marker gene is an antibiotic resistant gene, identifying the cell may comprise, for example, identifying a proliferation of the cell in a culture medium including an antibiotic. Also, the marker gene may be, for example, a fluorescent protein gene as described above. When the marker gene is the fluorescent protein gene, identifying the cell may comprise identifying the cell expressing fluorescence.

According to another aspect of the present invention, there is provided a method of isolating a cell where the target gene has been deleted. The method comprises, consists essentially of, or consists of introducing a cassette for deleting a target gene into a host cell; and isolating cells expressing fluorescence among cells where the cassette has been introduced.

The method may further comprise preparing a cassette for deleting a target gene, the cassette comprising (a) a promoter-specific homologous region, (b) a fluorescent protein gene operably linked to the promoter-specific homologous region, and (c) a gene-specific homologous region, which is located adjacent to 3′-end of the fluorescent protein gene.

Isolation of the cells may be performed by a flow cytometry analysis. The flow cytometry analysis may be, for example, fluorescence-activated cell sorting (FACS).

An efficient gene modification and selection are possible using the cassette according to an aspect of the present invention.

Additionally, an efficient gene modification and selection are possible by using the method of preparing a cell where a target gene has been deleted by using the cassette according to an aspect of the present invention.

Moreover, an efficient gene modification and selection are possible by using the method of isolating a cell where the target gene has been deleted according to an aspect of the present invention.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

### Example 1

### Preparing a ScADE2-Deletion Cassette

A deletion cassette was prepared for deleting a target gene, S. cerevisiae ADE2 (ScADE2).

A pBluescript II KS+ vector (Stratagene), including a gene for ampicillin resistance and a multi-cloning site, was excised using the restriction enzyme Pstl and then treated with Calf Intestinal Alkaline Phosphatase (CTAP, Fermentas). YEGa-MCS-CEN yeast vector (SEQ ID NO: 1) was excised using Pstl, thereby obtaining a DNA fragment (fragment 1) (SEQ ID NO: 2) having a size of 1,578 bp including an open reading frame (ORF) of ScURA3 and a S. cerevisiae GAL7 terminator (ScGAL7_{T}) for a correct termination of GFP protein. The fragment 1 and the pBluescript II KS+ vector treated with Pstl and CTAP were ligated to prepare a pBluTScURA vector. The pBluTScURA vector was excised by using the restriction enzyme EcoRI and treated with CTAP.

A pMOX-GFP vector (Park et al., Appl Environ Micobiol, 2007, 73: 5990-6000) was treated with EcoRI to obtain a DNA fragment (fragment 2) (SEQ ID NO: 3) having a size of 723 bp. The Fragment 2 and the pBluTScURA treated with EcoRI and CTAP were ligated to obtain a pBluTScURA-EGFP vector where a GFP gene was inserted before ScGAL7_{T} in a forward direction.

A primer 5UTR-ScADE2-GFP_{—}1F_X (SEQ ID NO: 4) and a primer yEGFPGIy_{—}2B_{—}44_HH (SEQ ID NO: 5) were prepared based on a S. cerevisiae genome database. After performing PCR using a pMOX-GFP vector as a template and the above-described primers, a PCR product was treated with the restriction enzymes XhoI/HpaI to obtain a DNA fragment (fragment 3) including a homologous region of 5′UTR of the ScADE2 gene (SEQ ID NO: 6). By ligating the fragment 3 and the pBluTScURA-EGFP excised with XhoI/HpaI, a pBluTScURA-Nade-EGFP vector was obtained.

After performing PCR using a YEGa-MCS-CEN vector as a template, a primer pScURA3_{—}1F_Bam_{—}41 (SEQ ID NO: 7), and a primer pScURA3_{—}2B_{—}43 (SEQ ID NO: 8), a PCR product was treated with the restriction enzymes BamHI/EcoRV to obtain a DNA fragment (fragment 4) (SEQ ID NO: 9) including a promoter of ScURA3. After performing PCR using genomic DNA of S. cerevisiae BY4742 strain as a template, a primer 3UTRScADE2_{—}1F_Bam_{—}44 primer (SEQ ID NO: 10), and a primer 3UTRScADE2_{—}2B_Sac_{—}42 (SEQ ID NO: 11), a PCR product was treated with the restriction enzymes BamHI/SacI to obtain a DNA fragment (fragment 5) including a 3′UTR homologous region of ScADE2 gene having a size of 150 bp (SEQ ID NO: 12).

The fragment 4, the fragment 5, and the pBluTScURA-Nade-EGFP vector treated with EcoRV/SacI were 3-piece ligated to finally obtain a pBluTScURA-NAde-EGFP-Cade vector including a ScADE2 deletion cassette. The vector was treated with the restriction enzymes XhoI/SacI, and a DNA fragment (SEQ ID NO: 13) having a size of 2,733 bp was used as a final deletion cassette.

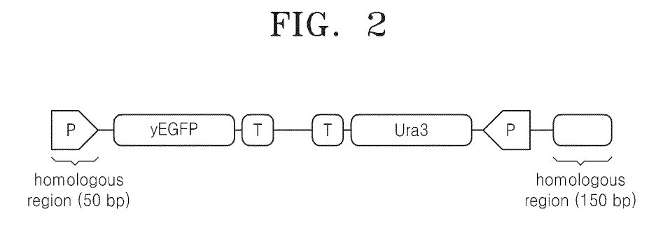

FIG. 2 schematically illustrates a deletion cassette for a deletion of the ScADE2 gene. The cassette includes a sequence for a promoter-specific homologous region having a size of 50 bp, a gene for yeast-enhanced green fluorescent protein (yEGFP) that is a marker gene, a transcription terminator of the yEGF, a gene for Ura3 (orotidine 5-phosphate decarboxylase), a promoter and a transcription terminator of Ura3, and a sequence of a gene-specific homologous region having a size of 150 bp.

### Example 2

### Introduction of the Cassette of Example 1

An S. cerevisiae BY4742 (MATa his3Δ1 leu2Δ0 lys2Δ0 ura3Δ0) strain was pre-cultivated in 3 mL of liquid YPD culture medium for 16 hours, inoculated in 50 mL of liquid YPD at an initial OD value of 0.4, and cultivated for 3 hours until the OD value reached 1. After centrifuging (3,000 rpm, 4° C., 5 min), cells were recovered and then washed once by using 20 mL of 1×TE (0.01 M Tris-HCl (pH 7.5), 1 mM EDTA (pH 8.0)), and a competent cell was prepared by adding 500 μL of 1×TE/LiAc. Thereafter, 100 μL of the competent cell, 10 μL of a DNA fragment of a gene-deletion cassette (approximately 0.5 μg), 100 μg/10 μL of salmon sperm DNA, 600 μL of PEG/LiAc (50% polyethylene glycol, 0.01 M Tris-HCl (pH 7.5), 1 mM of EDTA (pH 8.0), and 0.1 M of LiAc (pH 7.5)) were mixed and then stirred for 30 minutes at a temperature of 30° C. in a shaking incubator. 70 μL of DMSO was added to the solution, and the resultant was mixed and heat shocked for 15 minutes at a temperature of 42° C. After cooling on ice for 5 minutes, the solution was centrifuged at 3,000 rpm for a minute, and suspended in 100 μL of triple distilled water to prepare a suspension liquid. The suspension liquid was spread on an SC-URA selective medium (0.67% yeast nitrogen base without amino acid, 2% glucose, amino acid dropout mixture without uracil), then the medium was incubated for three days at a temperature of 30° C. to obtain cell colonies. 29 of the colonies were re-inoculated in the SC-URA selective medium.

### Example 3

### Identifying ScADE2-Deleted Cells

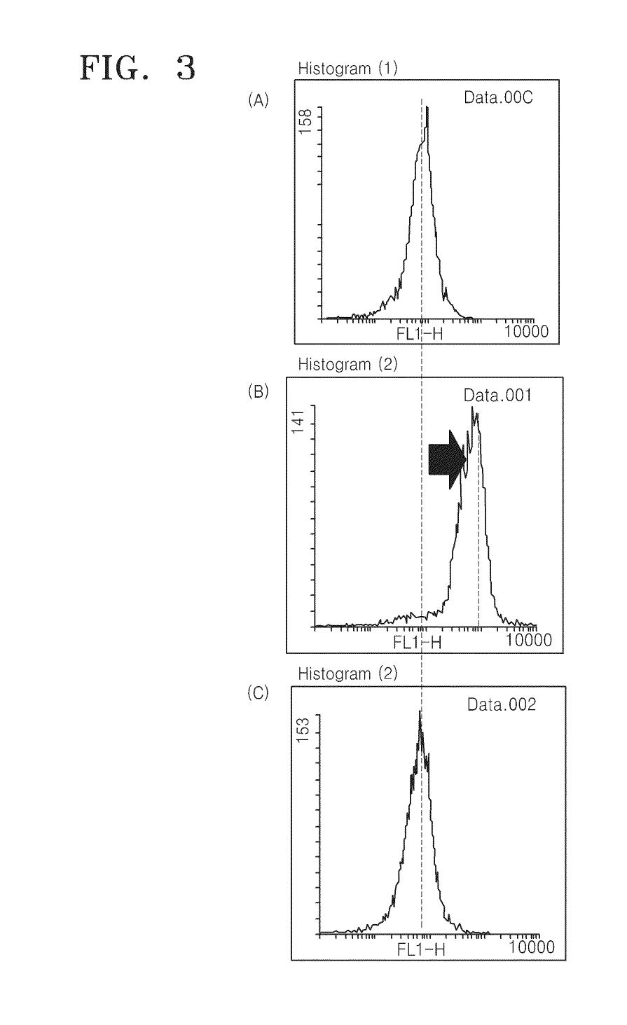

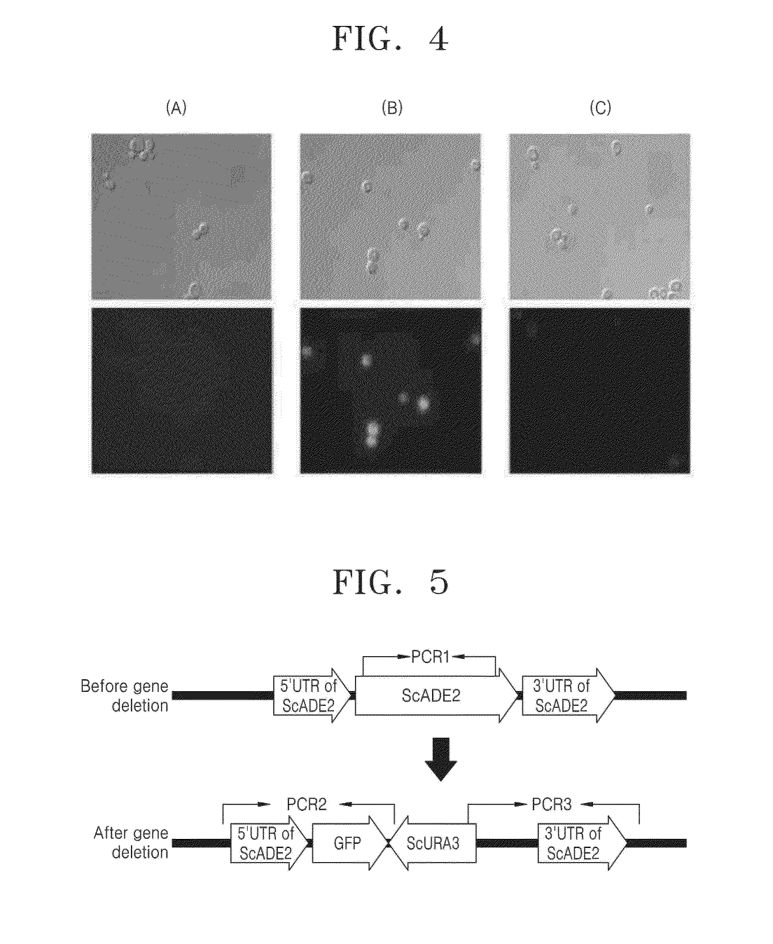

In order to isolate ScADE2-deleted cells from the 29 colonies obtained in Example 2, strains expressing GFP protein were selected using a flow cytometry. In a BD Facscaliber flow cytometry analyzer, a dichroic mirror (DM 56SP), a 90/10 beam splitter, and a 530/30 filter were used, and a 488 nm argon ion laser was irradiated to measure a fluorescence value at a fluorescence parameter FL. In 27 strains out of the 29 strains tested, a shift of a fluorescence peak was observed (FIG. 3B) when compared to a wild-type BY4742 strain (FIG. 3A). When wild type strain (FIG. 3A) and a strain that showed identical fluorescence value as the wild type strain (FIG. 3C) were analyzed by using a fluorescence microscope (Zeiss Axiophot epifluorescence microscope, Carl Zeiss, Germany), the strains did not show expression of a GFP protein (FIGS. 4A and 4C). Fluorescent signal was only detected in cells where the cassette has been targeted to the ScADE2 gene (FIG. 4B).

In order to confirm an occurrence of a ScADE2 gene deletion through a proper insertion of a deletion cassette in a GFP-expressing strain, 29 strains were subject to 3 different PCR studies. FIG. 5 illustrates a PCR analysis for identifying the ScADE2 gene deletion by using the GFP deletion cassette. In a first PCR study, a primer set Iden_ScADE2inside_{—}1F (SEQ ID NO: 14) and Iden_ScADE2inside_{—}2B (SEQ ID NO: 15) for amplification of ScADE2 ORF were used (PCR 1). In a second PCR study, a forward primer Iden_{—}5UTRScADE2_{—}1F (SEQ ID NO: 16) that attaches to the 5′UTR of ScADE2 that is located on the outer side of the cassette and a reverse primer ScURA3_C_{—}1F (SEQ ID NO: 17) that attaches to the 3′ region of ScURA3 ORF were used (PCR 2). In a third PCR study, a forward primer ScURA3_N_{—}2B (SEQ ID NO: 18) that attaches to the 5′ region of ScURA3 and a reverse primer Iden_{—}3UTRScADE2_{—}2B (SEQ ID NO: 19) that attaches to the 3′UTR of ScADE2 located outside of the cassette were used (PCR 3) to confirm an amplification.

As a result, the ScADE2 ORF was confirmed to be maintained in the case of a strain that had an identical fluorescence value as the wild-type strain in a flow cytometry analysis. For 4 strains of the 27 strains without ORF amplification, the amplification did not occur in the second PCR study, but occurred in the third PCR study. Thus, it was concluded that the cassette was inserted in only one direction.

As described above, 23 proper deletion strains were selected from 27 GFP expression strains by selecting deletion strains using the GFP deletion cassette and flow cytometry analysis. The results suggest that a selection of strains may be possible at an efficiency rate of about 85% only through a GFP expression. Accordingly, flow cytometry analysis was confirmed to be effective in selecting deletion strains by using the GFP deletion cassette.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A nucleic acid cassette for deleting a target gene, the cassette comprising
(a) a promoter-specific homologous region having a sequence identity to a portion of a promoter region of the target gene, wherein the degree of sequence identity is sufficient to drive homologous recombination therebetween,
(b) a marker gene operably linked to the promoter-specific homologous region, and
(c) a gene-specific homologous region adjacent to 3′-end of the marker gene and having a sequence identity to at least a portion of the target gene, wherein the degree of sequence identity is sufficient to drive homologous recombination therebetween.

2. The cassette of claim 1, wherein the portion of a promoter region of the target gene comprises a region of 40 to 150 nucleotides from 3′ end of the promoter.

3. The cassette of claim 1, wherein the marker gene is an antibiotic resistant gene or a fluorescent protein gene.

4. The cassette of claim 1, wherein the portion of the target gene comprises a region of 40 to 500 nucleotides of the target gene.

5. A method of preparing a cell where a target gene has been deleted, the method comprising:
introducing the cassette of claim 1 into a host cell; and
identifying a cell where the target gene has been deleted among cells where the cassette has been introduced by assaying for the expression of the marker gene.

6. The method of claim 5, further comprises preparing the cassette, wherein preparing the cassette comprises amplifying the marker gene using a polynucleotide comprising the marker gene as a template, a forward primer comprising a 5′-terminal region sequence of the marker gene and a sequence of the promoter-specific homologous region, and a reverse primer comprising a 3′-terminal region sequence of the marker gene and a sequence of the gene-specific homologous region.

7. The method of claim 5, wherein the host cell is yeast.

8. The method of claim 5, wherein the cassette introduced into the host cell is integrated into a chromosome of the host cell through a homologous recombination.

9. The method of claim 5, wherein the marker gene is an antibiotic resistant gene.

10. The method of claim 9, wherein identifying the cell where the target gene has been deleted comprises identifying cells proliferating in a culture medium comprising an antibiotic.

11. The method of claim 5, wherein the marker gene is a fluorescent protein gene.

12. The method of claim 11, wherein identifying the cell where the target gene has been deleted comprises identifying cells expressing fluorescence.

13. A method of isolating a cell in which a target gene has been deleted, the method comprising:
introducing the cassette of claim 1 into a host cell, wherein the marker gene encodes a fluorescent protein; and
isolating cells expressing fluorescence among cells where the cassette has been introduced.

14. The method of claim 13, wherein isolating the cell is performed by flow cytometry analysis.


SEQUENCE LISTING (19 sequences)

SEQ ID NO: 1 - DNA, 5285 bp, Artificial Sequence
  Synthetic (YEGa-MCS-CEN)
atgaccatgattacgaattaattcgagctcggtacccggggatccatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggaccagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcaagaattcagatctcgagaagcttgcatgcaactgcaggcggccgcggatcgatgtcgacttgaacggagtgacaatatatatatatatatatttaataatgacatcattatctgtaaatctgattcttaatgctattctagttatgtaagagtggtcctttccataaaaaaaaaaaaaaagaaaaaagaattttaggaatacaatgcagcttgtaagtaaaatctggaatattcatatcgccacaacttcttatgcttataaaagcactaatgcctgaatttatgttgaaaatatgtgtcacaaataaagaaactgtgacatctgacacatttccactttattgacaagaatagaatttctttaagtttcccctctagattatttattttcaaattttaggctctgttgaagtttattacgtagaaattcctacgatagttattagtcctaattggatgttgcagcaaggctcattgtcggtgtcgttatcgagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagct

SEQ ID NO: 2 - DNA, 1578 bp, Artificial Sequence
  Synthetic (YEGa-MCS-CEN_PstI)
ggcggccgcggatcgatgtcgacttgaacggagtgacaatatatatatatatatatttaataatgacatcattatctgtaaatctgattcttaatgctattctagttatgtaagagtggtcctttccataaaaaaaaaaaaaaagaaaaaagaattttaggaatacaatgcagcttgtaagtaaaatctggaatattcatatcgccacaacttcttatgcttataaaagcactaatgcctgaatttatgttgaaaatatgtgtcacaaataaagaaactgtgacatctgacacatttccactttattgacaagaatagaatttctttaagtttcccctctagattatttattttcaaattttaggctctgttgaagtttattacgtagaaattcctacgatagttattagtcctaattggatgttgcagcaaggctcattgtcggtgtcgttatcgagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgca

SEQ ID NO: 3 - DNA, 723 bp, Artificial Sequence
  Synthetic (pMOX_GFP_EcoRI)
aattcatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataag

SEQ ID NO: 4 - DNA, 80 bp, Artificial Sequence
  Synthetic (5UTR-ScADE2-GFP_1F_X)
gtcactcgagcctactataacaatcaagaaaaacaagaaaatcggacaaaacaatcaagtatgtctaaaggtgaagaatt

SEQ ID NO: 5 - DNA, 45 bp, Artificial Sequence
  Synthetic (yEGFPGly_2B_44_HH)
gcataagcttaccaccaccaccacctttgtacaattcatccatac

SEQ ID NO: 6 - DNA, 50 bp, Artificial Sequence
  Synthetic (ScADE2_5′UTR_homo)
cctactataacaatcaagaaaaacaagaaaatcggacaaaacaatcaagt

SEQ ID NO: 7 - DNA, 48 bp, Artificial Sequence
  Synthetic (pScURA3_1F_Bam_41)
catggatccaagcttgtatttaaattgtttcaattcaattcatcattt

SEQ ID NO: 8 - DNA, 18 bp, Artificial Sequence
  Synthetic (ScURA3_2B_43)
tgtgcattcgtaatgtct

SEQ ID NO: 9 - DNA, 590 bp, Artificial Sequence
  Synthetic (ScURA3_pro)
gcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcaca

SEQ ID NO: 10 - skipped
  Synthetic (3UTRScADE2_1F_Bam_44)

SEQ ID NO: 11 - DNA, 31 bp, Artificial Sequence
  Synthetic (3UTRScADE2_2B_Sac_42)
tacgagctctcttatgtatgaaattcttaaa

SEQ ID NO: 12 - DNA, 150 bp, Artificial Sequence
  Synthetic (ScADE2_3′UTR_homo)
tatataagtttattgatatacttgtacagcaaataattataaaatgatatacctattttttaggctttgttatgattacatcaaatgtggacttcatacatagaaatcaacgcttacaggtgtccttttttaagaatttcatacataaga

SEQ ID NO: 13 - DNA, 2733 bp, Artificial Sequence
  Synthetic (ScADE2_del_cassette)
gcctactataacaatcaagaaaaacaagaaaatcggacaaaacaatcaagtatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataagaattcctgcaggcggccgcggatcgatgtcgacttgaacggagtgacaatatatatatatatatatttaataatgacatcattatctgtaaatctgattcttaatgctattctagttatgtaagagtggtcctttccataaaaaaaaaaaaaaagaaaaaagaattttaggaatacaatgcagcttgtaagtaaaatctggaatattcatatcgccacaacttcttatgcttataaaagcactaatgcctgaatttatgttgaaaatatgtgtcacaaataaagaaactgtgacatctgacacatttccactttattgacaagaatagaatttctttaagtttcccctctagattatttattttcaaattttaggctctgttgaagtttattacgtagaaattcctacgatagttattagtcctaattggatgttgcagcaaggctcattgtcggtgtcgttatcgagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaacaatttaaatacaagcttggatcctatataagtttattgatatacttgtacagcaaataattataaaatgatatacctattttttaggctttgttatgattacatcaaatgtggacttcatacatagaaatcaacgcttacaggtgtccttttttaagaatttcatacataagag

SEQ ID NO: 14 - DNA, 18 bp, Artificial Sequence
  Synthetic (Iden_ScADE2inside_1F)
tgttgaggcagcaaacag

SEQ ID NO: 15 - DNA, 18 bp, Artificial Sequence
  Synthetic (Iden_ScADE2inside_2B)
cgcagcgttcgtactatt

SEQ ID NO: 16 - DNA, 18 bp, Artificial Sequence
  Synthetic (Iden_5UTRScADE2_1F)
ttgcatggctacgaaccg

SEQ ID NO: 17 - DNA, 18 bp, Artificial Sequence
  Synthetic (ScURA3_C_1F)
agaagatgcggccagcaa

SEQ ID NO: 18 - DNA, 19 bp, Artificial Sequence
  Synthetic ( ScURA3_N_2B)
gcagcacgttccttatatg

SEQ ID NO: 19 - DNA, 18 bp, Artificial Sequence
  Synthetic (Iden_3UTRScADE2_2B)
cttgcttcttgttactgg